(19) United States
(12) Patent Application Publication
(10) Pub. No.: US 2015/0224161 A1
(43) Pub. Date: Aug. 13, 2015

(54) METHODS AND USES OF AN EXTRACT FROM OLIVE LEAF IN MANAGEMENT OF TYPE 2 DIABETES

(71) Applicant: APIMED MEDICAL HONEY LIMITED, Paengaroa (NZ)

(72) Inventors: Martin De Bock, Grafton (NZ); Stephen Hodgkinson, Grafton (NZ); Wayne Cutfield, Grafton (NZ); Ralf Christian Schlothauer, Grafton (NZ)

(21) Appl. No.: 14/426,140

(22) PCT Filed: Sep. 4, 2013
(86) PCT No.: PCT/NZ2013/000159

§ 371 Date: Mar. 4, 2015

(30) Foreign Application Priority Data
Sep. 5, 2012 (NZ) 602245

(51) Int. Cl. A61K 36/63; A23L 1/29; A61K 9/48; A61K 31/7048; A61K 31/05

(52) U.S. Cl. CPC A61K 36/63; A61K 31/7048; A61K 31/05; A61K 9/48; A23L 1/293; A23V 2002/00

(57) ABSTRACT

Described herein are methods and uses of oleuropein and hydroxytyrosol in a variety of applications to prevent, reduce symptoms of and treat conditions related to insulin sensitivity including type 2 diabetes.

### TECHNICAL FIELD

Described herein are methods and uses of a therapeutically effective amount of a water based extract derived from olive (Olea europaea) leaves are described to assist with management of and reduce the symptoms of type 2 diabetes in a patient or subject.

### BACKGROUND ART

It is estimated that 20-50% of the European population use complementary and alternative therapy to treat disease or to help prevent disease onset. According to one reference, one third of type 2 diabetics in the USA actively use complementary and alternative medicine to treat their condition, for which most remedies carry no scientific evidence. Folk medicine using olive plants to treat diabetes has existed for centuries, and ancient references to the medical use of the olive plant can be found in the bible. Recently, the medicinal properties of olive products have focussed on olive polyphenols—particularly oleuropein (OL) and hydroxytyrosol (HT), which in animal and in-vitro studies have been shown to have anti-oxidant, hypoglycaemic, antihypertensive, antimicrobial, and anti-atherosclerotic properties.

Oleuropein is a bitter secoiridoid glycoside identified as the most abundant and major bipolyphenolic compound in olive leaves while monomeric phenols denote the minor compounds. In total 54 phenolic compounds have been found in live leaf extract (OLE) with 46 of them tentatively identified. Although many biophenols have been found in OLE the strongest antioxidant activities has been attributed to oleuropein. The bioactivity of this compound is diverse and its metabolites such as the secoiridoid hydroxytyrosol, are potent antioxidant and free radical scavengers. The structures of oleuropein and hydroxytyrosol are as shown below:

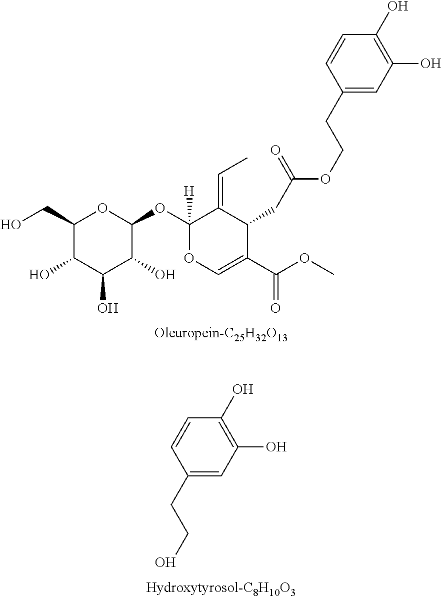 [SMILES: C/C=C1/[C@H](O[C@@H]2O[C@H](CO)[C@@H](O)[C@H](O)[C@H]2O)OC=C(C(=O)OC)[C@H]1CC(=O)OCCc1ccc(O)c(O)c1 ; OCCc1ccc(O)c(O)c1]

Other classes of phenolic compounds are present in OLE, including verbacoside, flavonoids (luteolin-7-glucoside, apigenin-7-O-glucoside, rutin and luteolin 4′-O-glucoside) and oleuroside. Oleuropein with its many biological activities is a renowned and proven significant source of bioactivity offering great pharmacological potential in the nutraceutical, cosmetic and pharmaceutical industries.

Polyphenols are found in most edible plants, and are considered to deliver the health benefits of chocolate, coffee, green tea, and red wine. Paralleling the growth in scientific knowledge in olive polyphenols, the olive nutraceutical market is expanding. As the concentration of olive polyphenols is far more potent in olive leaves compared to the fruit or olive oil, this once discarded by-product of tree pruning is now a valuable commodity.

While the cardiovascular health benefits of a Mediterranean diet rich in olive oil is well established, human clinical studies examining cardiovascular disease risk factors during supplementation with olive polyphenols are scarce and often have methodological flaws, or are contradictory. The European Food Safety Authority has endorsed the health claim that “the consumption of olive oil polyphenols contributes to the protection of blood lipids to oxidative damage”, while rejecting several others—however no claims were made on the basis of glucose homeostasis. Hence, while in-vitro and animal model work has provided a backdrop for therapeutic potential, there remains a lack of knowledge in this area investigating health effects on human subjects. The physician is left with the difficult task of counselling patients about complementary medicine without corroborating evidence that usually allows decisions to be made with confidence.

It should be appreciated that a composition formulated for oral administration to a patient that prevents, treats and/or reduces the symptoms of insulin sensitivity and related diseases such as type 2 diabetes may have considerable value or at least provide the public with a choice.

Further aspects and advantages of the process and product will become apparent from the description below that is given by way of example only.

### SUMMARY

Described herein are methods and uses of a therapeutically effective amount of a water based extract derived from olive (Olea europaea) leaves are described to assist with management of and reduce the symptoms of type 2 diabetes in a patient.

In a first aspect, there is provided a method of reducing the symptoms associated with type 2 diabetes in a subject, by the step of oral administration to the subject of a therapeutically effective amount of a water based extract derived from olive (Olea europaea) leaves, wherein the compounds oleuropein and hydroxytyrosol present in the extract at a ratio of approximately 0.5 to 2.0 parts hydroxytyrosol to approximately 5 parts oleuropein.

In a second aspect, there is provided the use of a water based extract derived from olive (Olea europaea) leaves in the manufacture of a composition formulated for oral administration to a subject to reduce the symptoms associated with type 2 diabetes, wherein the extract contains wherein the extract contains a therapeutically effective amount of the compounds oleuropein and hydroxytyrosol in the extract at a ratio of approximately 0.5 to 2.0 parts hydroxytyrosol to approximately 5 parts oleuropein.

The inventors have identified that a an olive leaf extract composition containing oleuropein, hydroxytyrosol and other phenolics, when administered orally, may have significant benefits in treating, reducing the symptoms of and/or preventing type 2 diabetes, in both at risk subjects and those already with the disease. In trials completed by the inventors, improved insulin action was shown after 12 weeks of olive leaf extract supplementation in overweight middle-aged men at risk of developing future metabolic syndrome. This finding was independent of lifestyle factors, as no changes were observed in diet or physical activity or change in BMI or fat distribution. Trials in patients already with the disease also show positive results with patients being able to avoid more drastic intervention medications such as insulin injections, lowered medication doses and general improvements in health and well being. As may be appreciated, even a reduction in the symptoms associated with type 2 diabetes may be of considerable benefit given the debilitating effects of the disease and it's cost on both the patients and society in treating these patients as well.

A further advantage of the above methods and uses include use of naturally occurring compounds in addressing the considerable problem of insulin sensitivity. The extract used is able to be grown via sustainable methods and the extract appears to have minimal if any side effects unlike some medication drugs currently used to treat type 2 diabetes. Further, the patient may administer the extract with minimal fuss unlike for example, insulin injections required in treatment of poorly managed type 2 diabetes.

### BRIEF DESCRIPTION OF THE DRAWINGS

Further aspects of the methods and uses will become apparent from the following description that is given by way of example only and with reference to the accompanying drawings in which:

FIG. 1 illustrates a trial plan described in Example 1;

FIG. 2 illustrates the measured insulin sensitivity (ISI) of subjects tested during a trial to illustrate the efficacy of the composition; and,

FIG. 3 illustrates the measured pancreatic β-cell responsiveness of a subject tested during a trial to further illustrate the efficacy of the composition.

### DETAILED DESCRIPTION

As noted above, methods and uses of a therapeutically effective amount of a water based extract derived from olive (Olea europaea) leaves are described to assist with management of and reduce the symptoms of type 2 diabetes in a patient.

For the purposes of this specification, the term ‘about’ or ‘approximately’ and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1% to a reference quantity, level, degree, value, number, frequency, percentage, dimension, size, amount, weight or length.

The term ‘substantially’ or grammatical variations thereof refers to at least about 50%, for example 75%, 85%, 95% or 98%.

The term ‘comprise’ and grammatical variations thereof shall have an inclusive meaning—i.e. that it will be taken to mean an inclusion of not only the listed components it directly references, but also other non-specified components or elements.

The term ‘reducing the symptoms associated with’ or grammatical variations thereof refers to preventing or treating a subject: (a) with type 2 diabetes or (b) at risk of developing type 2 diabetes.

The terms ‘prevent’ or ‘treat’ or grammatical variations thereof refers to either stopping, improving or at least minimising the effects of symptoms associated with type 2 diabetes.

The term ‘therapeutically effective amount’ or grammatical variations thereof with reference to an amount or dosage of the composition described herein refers to an amount of a composition which is sufficient to effectively cause the described action such as preventing, treating or reducing a condition, a disease or symptoms.

The term ‘condition’ refers to an abnormality in the physical state of the body as a whole or on of its parts. For an example ‘disease’ refers to a pathological condition of a part, organ, or system of an organism resulting from various causes, such as an infection, genetic defect, environmental stress, weight or diet, and is typically characterised by an identifiable group of signs or symptoms.

The term ‘type 2 diabetes’ refers to diabetes mellitus type 2 formally known as non-insulin-dependent diabetes mellitus (NIDDM) or adult-onset diabetes, being a metabolic disorder characterised by high blood glucose in the context of insulin resistance and relative insulin deficiency.

The terms ‘patient’ and ‘subject’ or grammatical variations thereof may be used interchangeably to refer to the animal to which a composition or medicament is administered.

In a first aspect, there is provided a method of reducing the symptoms associated with type 2 diabetes in a subject, by the step of oral administration to the subject of a therapeutically effective amount of a water based extract derived from olive (Olea europaea) leaves, wherein the compounds oleuropein and hydroxytyrosol present in the extract at a ratio of approximately 0.5 to 2.0 parts hydroxytyrosol to approximately 5 parts oleuropein.

In a second aspect, there is provided the use of a water based extract derived from olive (Olea europaea) leaves in the manufacture of a composition formulated for oral administration to a subject to reduce the symptoms associated with type 2 diabetes, wherein the extract contains wherein the extract contains a therapeutically effective amount of the compounds oleuropein and hydroxytyrosol in the extract at a ratio of approximately 0.5 to 2.0 parts hydroxytyrosol to approximately 5 parts oleuropein.

The inventors have identified that a an olive leaf extract composition containing oleuropein, hydroxytyrosol and other phenolics, when administered orally, may have significant benefits in treating, reducing the symptoms of and/or preventing type 2 diabetes, in both at risk subjects and those already with the disease. In trials completed by the inventors, improved insulin action was shown after 12 weeks of olive leaf extract supplementation in overweight middle-aged men at risk of developing future metabolic syndrome. This finding was independent of lifestyle factors, as no changes were observed in diet or physical activity or change in BMI or fat distribution. Trials in patients already with the disease also show positive results with patients being able to avoid more drastic intervention medications such as insulin injections, lowered medication doses and general improvements in health and well being. As may be appreciated, even a reduction in the symptoms associated with type 2 diabetes may be of considerable benefit given the debilitating effects of the disease and it's cost on both the patients and society in treating these patients as well.

Without being bound by theory, the applicant understands that the mechanism may derive from the ability of the olive leaf extract inhibit amylase activity and in doing so allow carbohydrates to reach the ileum and in doing so trigger the ileal brake. Olive leaf extract also appears to have GLP agonist activity in the body. These mechanisms give the patient a feeling of satiety sending signals to the patient's brain to eat less and/or instead give a feeling of being ‘full’. The compounds in the olive leaf extract also appear to increase insulin sensitivity and pancreatic β-cell secretion capacity. In the applicant's work, pre-diabetic and diabetic patients see a corresponding drop in glucose levels in the blood when taking olive leaf extract. Another observation supporting the above proposed mechanism is that patients diagnosed with diabetes develop a feeling of being less hungry—a feeling at odds with the fact the same patients are more energetic and burning more energy through daily movement but perhaps explained by the fact the glucose is better able to be utilised by the body for energy when on olive leaf extract.

Treatment as noted above may result in increased glucose tolerance in the subject relative to a measured glucose tolerance in the patient if untreated.

In the above methods and uses, administration of the composition may result in an improved insulin action. Improved action may be at least a 1, or 2, or 3, or 4, or 5, or 6, or 7, or 8, or 9, or 10, or 11, or 12, or 13, or 14, or 15, or 16 or 17, or 18, or 19, or 20% improvement in insulin action compared to an untreated subject.

In the above methods and uses, administration of the composition may result an improvement in insulin sensitivity (ISI). Treatment may result in an at least 1 or 2, or 3, or 4, or 5, or 6, or 7, or 8, or 9, or 10, or 11, or 12, or 13, or 14, or 15, or 16 or 17, or 18, or 19, or 20% increase in insulin sensitivity in the subject relative to measured insulin sensitivity in the subject if untreated.

In the above methods and uses, administration of the composition may result an improvement in blood sugar levels. Treatment may result in reduced HbA1 c levels in the subject relative to measured blood sugar levels in the subject if untreated. Treatment may result in a reduction in HbA1c levels of at least 1, or 2, or 3, or 4, or 5, or 6, or 7, or 8, or 9, or 10, or 11, or 12, or 13, or 14, or 15%.

In the above methods and uses, administration of the composition may result in improved insulin excretion. Treatment may result in an at least 5, or 6, or 7, or 8, or 9, or 10, or 11, or 12, or 13, or 14, or 15, or 16 or 17, or 18, or 19, or 20, or 21, or 22, or 23, or 24, or 25, or 26, or 27, or 28, or 29, or 30% increase in insulin excretion relative to measured insulin excretion in the subject if untreated.

In the above methods and uses, administration of the composition may result in increased pancreatic (3-cell secretion capacity. Treatment may result in an at least a 5, or 6, or 7, or 8, or 9, or 10, or 11, or 12, or 13, or 14, or 15, or 16 or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40% increase pancreatic (3-cell secretion capacity relative to measured pancreatic (3-cell secretion capacity in the subject if untreated.

In the above methods and uses, administration of the composition may result in increased IL-6 cytokine production. Treatment may result in an at least 5, or 6, or 7, or 8, or 9, or 10, or 11, or 12, or 13, or 14, or 15, or 16 or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40% increase in IL-6 cytokine production relative to measured IL-6 cytokine production in the subject if untreated. The inventors in trial results have identified an acute rise in IL-6, a pro-inflammatory cytokine. IL-6 functions differently depending on the tissue it acts upon and concentration dynamics. Acute increases improve insulin regulated glucose metabolism in the muscle, while chronically low grade increased values are associated with a pro-inflammatory insulin resistant state in the liver. Mechanisms proposed through which IL-6 improves insulin sensitivity are: increased GLUT4 translocation in an AMP-activated protein kinase dependent manner and, activation of IRS/Akt signalling. The inventors speculate based on their findings that the composition described acts to improve insulin sensitivity and glucose uptake at least in part through IL-6 production.

Administration in the above methods and uses may further result in increased IGFBPI and IGFBP2 cytokine production. A trend was observed in trials completed by the inventor's towards increased IGFBP1 and IGFBP2. Increased IGFBP2 protects against the development of obesity and improves insulin sensitivity, and higher IGFBP1 is associated with improved insulin action.

As may be appreciated, the above actions are comparable to medications commonly used to treat diabetics, for example metformin, thiazolodinediones, and GLP-1 agonists. These medications may however have detrimental side effects hence, the above composition may be beneficial if only to provide an alternative treatment means with minimal if any side effects. At the very least, the methods and uses described herein may be used in conjunction with traditional medications as a means to enhance the treatment and/or amelioration effects. Further, since the methods and uses herein also show preventative capabilities and minimal if any side effects, the methods and uses described may be used to prevent type 2 diabetes or at least slow the on-set—existing medications due to side effects may be better suited for use in treatment rather than prevention.

When administered for prevention, the subject to whom the composition is administered may have one or more risk factors associated with type 2 diabetes. A risk factor may be a body mass index (BMI) greater than 25.0 or an overweight to obese weight. A risk factor may be a family history of type 2 diabetes. A further risk factor may be poor diet and lack of exercise by the subject.

Further, in respect of prevention, for impaired glucose tolerance to progress into diabetes, patients need to become both insulin resistant, and lose pancreatic β-cell secretion capacity. The inventors have showed improvement in pancreatic β-cell secretion capacity by use of the methods and uses described. Therefore, as both insulin resistance and pancreatic β-cell secretion capacity are improved, it is envisaged by the inventors that administration of the composition described may protect against the progression of impaired glucose tolerance to type 2 diabetes. These findings reflect the counter intuitive observations of patients noting they eat less yet have more energy reinforcing the findings that OLE treatment not only increases insulin sensitivity and production, but also increases the effectiveness of the insulin in dealing with excess glucose.

The hydroxytyrosol if present may be glucoronidated. Glucoronidation is the addition of a glucuronic acid to the hydroxytyrosol substrate. This step may be undertaken to make the hydroxytyrosol more bioavailable by making hydroxytyrosol more water soluble and/or more easily transported around the body. Hydroxytyrosol is not in itself particularly water soluble hence gluronidation, which increases solubility, may provide a useful means to increase the bioavailability.

As noted above, the composition used in the methods and uses above may be a water based extract derived from olive (Olea europaea) leaves.

The extraction steps undertaken to form the extract may be critical to the success of the composition in achieving the methods and uses described above.

The inventors have determined that it is preferable to obtain a ratio of OL to HT in the levels defined above. HT is a breakdown product of OL and without being certain of the mechanism, maintaining the OL form of the phenol in as greater a concentration as possible may be beneficial. Achieving such rations and maximising OL concentration appears to be an optimum based on method of extraction, the amount of leaf chopping or cutting and the duration of extraction.

Further, as noted above, the extract may be produced via a water based extraction method. While other solvent based extraction methods may be undertaken, water based methods appear to produce commercially significant amounts of the desired active compounds and retains the activity of the compounds.

In one embodiment, the olive leaf extract is produced by immersing the leaves in water at a temperature of at least approximately 50, or 55, or 60, or 65, or 70, or 75, or 80, or 85, or 90, or 95, or 100° C. Immersion may occur for a time period of at least 30, or 40, or 50, or 60, or 70, or 80, or 90, or 100, or 110, or 120, or 130, or 140, or 150, or 160, or 170, or 180 minutes. The form of mixing may be range from simple immersion of the leaves in water through to various types of wash, multiple washing, counter current extractions etc. Alternatively, extraction may be completed by blanching the leaves in steam for a period of time. In either case, the resulting water or condensate contains the active compounds desired and the solid leaf content may be separated from the liquid post extraction.

Also as noted above, there is an optimum in leaf size for the extraction step. Uncut whole leaves represent the most stable form of the actives however, without cutting enzymes that release the phenols such as oleuropein OL and hydroxytyrosol HT are less active and the speed and degree of extraction takes longer. Conversely, if the leaves are milled to a small size, too much enzyme activity occurs and the amount of OL dramatically reduces. In one embodiment, the leaves prior to extraction are chopped to a size of less than or equal to 15, or 14, or 13, or 12, or 11, or 10, or 9, or 8, or 7, or 6, or 5mm.

In one embodiment, the leaves may be processed and stabilised within at least 24, or 21, or 18, or 15, or 12, or 9, or 6, or 5, or 4, or 3, or 2, or 1 hour of picking/harvest. Freshness of the leaves may be critical to obtaining the desired activity of oleuropein and/or hydroxytyrosol. From the applicant's experience, the active compounds such as OL rapidly decrease in concentration over time once the leaves are picked from the tree.

Steps may be undertaken to stabilise the OL and/or HT in the composition before, during, or after extraction. In one embodiment, the compounds may be stabilised by reducing the water activity. Oleuropein in particular, is water sensitive and in high water activity environments quickly converts to other compounds. Removal of water activity halts this conversion. Methods to reduce the water activity may include drying or may include mixing the compounds with a hydrophilic carrier. The hydrophilic carrier may be sugars with a low osmolarity including but not limited to: glycerin, honey and combinations thereof. Inert oils such as safflower oil may also be used as carriers instead of sugars or in addition to sugars to reduce the water activity.

The composition may further include a therapeutically effective amount of oleanolic acid. Oleanic acid is also an amylase inhibitor along with at least oleuropein hence this may be the reason why oleanic acid may also assist. As may be appreciated, olive leaf extracts contain a variety of compounds and, other compounds besides the more prominent ones of oleuropein and/or hydroxytyrosol, may also be present including oleanic acid.

The composition in the above methods and uses may be formulated in various means. In one embodiment, the composition may be formulated as a liquid. Alternatively, the composition may be formulated as a powder, tablet or capsule. In further embodiments, the composition may be formulated as, or within a food, one example being a bread fortified with the composition described above. In a yet further embodiment, the composition may be formulated as, or within, a drink or beverage.

As noted above, the composition used in the above methods and uses may have both oleuropein and hydroxytyrosol present at a ratio of approximately 0.5 to 2.0 parts hydroxytyrosol to approximately 5 parts oleuropein.

The composition may be administered at a rate of at least 10, or 15, or 20, or 25, or 30, or 35, or 40, or 45, or 50, or 55, or 60, or 65, or 70, or 75, or 80, or 85, or 90, or 95, or 100, or 110, or 120, or 130, or 140, or 150mg oleuropein per day.

The composition may be administered at a rate of at least 0.1, or 0.2 or 0.3, or 0.4 or 0.5, or 1, 2, or 3, or 4, or 5, or 6, or 7, or 8, or 9, or 10, or 15, or 20, or 25, or 30, or 35, or 40mg hydroxytyrosol per day.

In one embodiment, the composition may be administered to the subject at a rate of approximately 30-150 mg oleuropein per day and approximately 0.3-40 mg hydroxytyrosol per day. In a yet further embodiment, the composition may be administered at a rate of 50 mg oleuropein per day and approximately 10 mg hydroxytyrosol.

The subject as described above may be a human in need thereof.

As noted above, advantages of the above include use of naturally occurring active compounds in addressing the considerable issues regarding insulin sensitivity. At one end of the spectrum is the ability to treat diabetes or related aspects of diabetes such as treating early signs of diabetes onset. There is an existing population of diabetes-effected patients needing treatment options using validated and scientifically proven remedies. At the other end of the spectrum is the ability to prevent, stop or at least slow the onset of diabetes or related symptoms. This option is rarely discussed in the art yet the inventors have proven considerable prevention benefits. At the very least, the methods and uses above may support existing medication treatments and may lower the dose required of such medications thereby reducing the potential side effects of existing medications.

The embodiments described above may also be said broadly to consist in the parts, elements and features referred to or indicated in the specification of the application, individually or collectively, and any or all combinations of any two or more said parts, elements or features, and where specific integers are mentioned herein which have known equivalents in the art to which the embodiments relates, such known equivalents are deemed to be incorporated herein as of individually set forth.

Where specific integers are mentioned herein which have known equivalents in the art to which this invention relates, such known equivalents are deemed to be incorporated herein as if individually set forth.

### WORKING EXAMPLES

The above described methods and uses are now described by reference to trials completed by to investigate the methods and uses.

### Example 1

### Methods:

### Study Population and Recruitment

Males aged 35-55 years were recruited by newspaper advertising for entry into the trial in

February 2010. Participants were required to have a BMI between 25 and 30 kg/m2. Exclusion criteria were smokers, those on medications likely to affect insulin sensitivity, or those with diabetes. Those taking anti-hypertensive or lipid lowering medications were included but were required to have been on a stable dose for at least 6 months prior to starting the study and encouraged not to change dose throughout the study.

### Study Design

The design was a randomized, double blind, placebo controlled, crossover trial. Participants were randomized to receive olive leaf extract or placebo for 12 weeks. Randomization and allocation to trial group were done using computer random number generation. After a 6 week washout phase participants crossed over to take the opposite intervention. Participants were assessed at baseline, and at the end of each intervention phase. Before each assessment participants were instructed to fast for greater than 8 hours, and avoid strenuous activity for 24 hours. Participants began assessments between 0630 and 0830 hrs. Throughout the whole study period participants were advised to not change their lifestyle in terms of diet or exercise. All clinical assessments were carried out at the Maurice & Agnes Paykel Clinical Research Unit (Liggins Institute, University of Auckland). Ethics was granted by the Northern Regional Y ethics committee, and the trial was registered at the Australasian Clinical Trial Registry (#00336317).

### Intervention

Capsules containing olive leaf extract (OLE) and placebo were used. The OLE product was a commercially available product sold by Comvita Ltd. Participants were instructed to take four capsules as a single dose once a day with a glass of water. The polyphenol dose in four OLE capsules was 51.1 mg of oleuropein and 9.7 mg of hydroxytyrosol as was independently verified by Conmac Laboratory Services (Queensland, Australia), and was suspended in safflower oil 672.5 mg. Placebo capsules were filled with 900 mg safflower oil and glycerin (an inert oil). Placebo and active capsules were identical in appearance, and both were odourless.

Primary and Secondary Outcomes and Assessments

The primary outcome assessed was glucose metabolism. Insulin sensitivity (ISI) was assessed by a 75 g oral glucose tolerance test (OGTT) using the Matsuda method. The Matsuda method has a strong correlation with the gold standard hyperinsulinemic euglycaemic clamp (r=0.77), and excellent reproducibility during multiple measures. Pancreatic β-cell responsiveness was measured by the oral glucose disposition index (DIₒ), and is predictive for future diabetes development. To search for mechanisms, cytokines that are known to influence glucose metabolism were measured: IGF-1, IGF2, IGFBPI, IGFBP2, IGFBP3, CRP, TNF-α, IL-6 and IL-8.

Secondary outcomes focused on other metabolic disease risk factors; lipid profiles, ambulatory blood pressure, body composition by whole-body dual-energy x-ray absorptiometry, and carotid intimal thickness (cIMT) by ultrasound as a direct measurement of atherosclerosis. Lifestyle factors were recorded with a 3 day itemised food diary, and a 7 day physical activity recall using the International Physical Activity Questionnaire (IPAQ).

Subjective measures of wellbeing were assessed by the Medical Outcomes Study Short Form (SF-36: New Zealand/Australia adaptation). The SF-36 is a validated tool that measures perception of health on eight multi-item dimensions covering functional status, wellbeing and overall evaluation of health.

### Sample Size

Our power calculation was based upon a known mean adult Matsuda ISI of 15.6 ±8.7 (mean +1-SD). To detect a 25% difference in ISI after the treatment periods with and without OLE required 42 subjects longitudinally studied with 80% power and a significance value of 0.05. This assumes a correlation of 0.5 between measurements on the same person. We aimed to recruit 50 subjects assuming a 10% drop out during the study.

### Statistical Analysis:

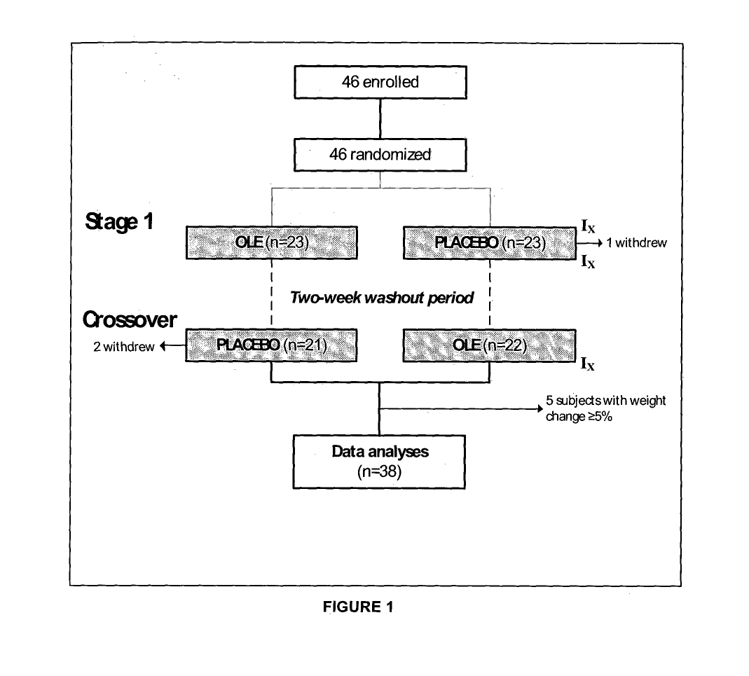

Crossover trial data were analysed in SAS v.9.2 (SAS Institute, Cary, NC, USA) using a mixed model design based on repeated measures, with the basic model including treatment (placebo vs OLE), treatment sequence (placebo→OLE vs OLE→Placebo), and treatment phase (Stage 1 vs Crossover; see FIG. 1) as factors, and the baseline reference sample as a co-variate. In addition, the model accounted for possible between-subject and between-period variations: IPAQ scores, age, and percentage body fat (from DXA scans). Importantly, statistical analyses were carried out on encoded data, such that the analyst (JGBD) was ‘blinded’ to treatment. The Johnson transformation was adopted when necessary to stabilize the variance in outcome responses. Data are expressed as means±SEM.

### Results:

### Recruitment:

Forty-six volunteers meeting inclusion criteria were enrolled into the trial. Four participants were on cholesterol lowering medication, three were on antihypertensives, and two were on both.

One participant dropped out of the study during stage 1 (due to injury), two withdrew after cross over (one was lost to follow up, another due to developing acne). All three that withdrew were taking placebo at the time (FIG. 1). Five participants had a greater than 5% weight change during the trial, implicating significant lifestyle change that would influence results and were subsequently not included in statistical analysis. This left 38 participants available for statistical analysis.

### Primary Outcome:

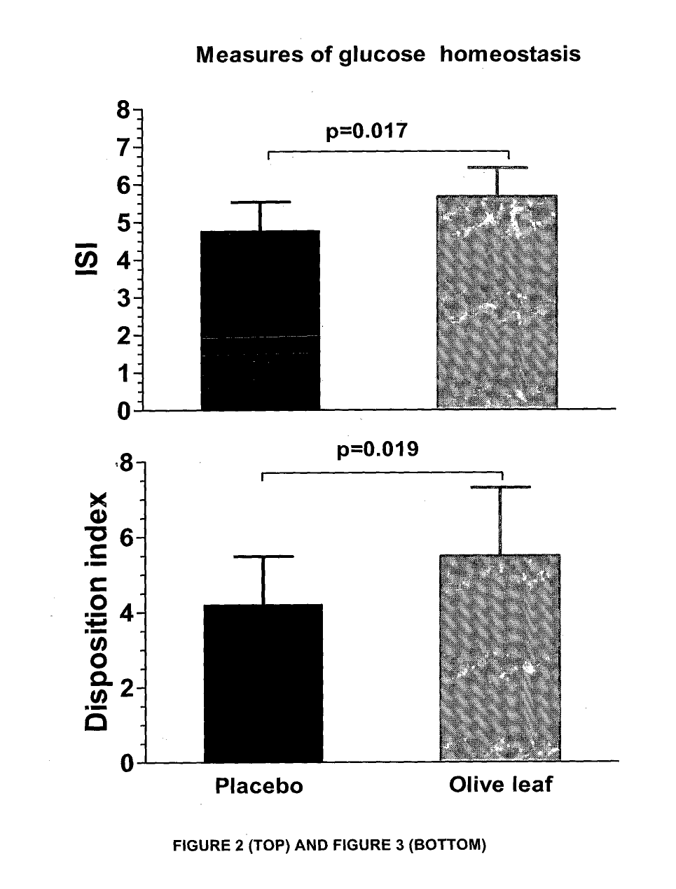

After OLE supplementation insulin sensitivity ISI improved by 19% (p =0.017), (FIG. 2). There was 30% improvement in pancreatic β-cell responsiveness (p=0.019), (FIG. 3). There were no differences in fasting glucose or insulin levels (data not shown). There was an acute increase in IL-6 while on olive leaf extract (p=0.047) (Table 1). There were no observed changes in IL-8 or TNF-a, or ultra sensitive CRP. While there were no differences in IGF-1, IGF-2, or IGFBP3, we observed a trend for increased IGFBP1 (p=0.067) and IGFBP2 (p=0.079) after OLE supplementation (Table 1).

Secondary Outcomes:

Laboratory outcomes are shown in Table 1. There were no changes in lipid profile ambulatory blood pressure, carotid intimal thickness or body composition. There was no statistically significant change across the three food diaries submitted by each participant. There were no differences in mean daily energy intakes or macronutrient contribution throughout the study. There was no change in physical activity over the study period as assessed by the IPAQ. There were no significant changes in health wellbeing assessments.

### Compliance and Safety:

Compliance was measure by counting capsules in returned containers. Compliance was universally excellent with no participants missing more than 3 days of capsules. Only once adverse event was reported; a flare up of acne. The participant withdrew from the study. After un-blinding, it was found that that the participant was receiving placebo. Liver function tests were monitored and there was no difference in AST, or ALT. There was small but clinically insignificant increase in ALP (47.2 vs 50.9units p=0.003) and decrease in GGT (33.0 vs 27.9 units p=0.0005) on OLE as compared to placebo.

TABLE 1
Biochemical measures following a 12-week supplementation
with olive leaf extract or placebo. Data are adjusted means
from multivariate models, and 95% confidence intervals.

| | Placebo | | Olive Leaf | | |
|---|---|---|---|---|---|
| Variable | Mean | 95% CI | Mean | 95% CI | p-value |
| Hormones | | | | | |
| IGF1 | 8.88 | 8.45-9.31 | 9.16 | 8.71-9.62 | 0.11 |
| IGF2 | 1.78 | 1.71-1.85 | 1.76 | 1.70-1.82 | 0.45 |
| IGFBP1 | 1.34 | 1.02-1.76 | 1.55 | 1.23-1.96 | 0.067 |
| IGFBP2 | 139 | 121-159 | 152 | 133-174 | 0.079 |
| IGFBP3 | 2295 | 2158-2440 | 2284 | 2175-2397 | 0.86 |
| Plasma | | | | | |
| Chol (mmol/l) | 4.53 | 4.32-4.74 | 4.73 | 4.52-4.95 | 0.054 |
| LDL (mmol/l) | 3.02 | 2.82-3.23 | 3.09 | 2.92-3.27 | 0.37 |
| HDL (mmol/l) | 1.05 | 0.99-1.10 | 1.04 | 0.98-1.10 | 0.76 |
| Proteins | | | | | |
| Interleukin-6 | 0.623 | 0.476-0.816 | 0.762 | 0.599-0.971 | 0.047 |
| Interleukin-8 | 1.80 | 1.62-1.99 | 1.86 | 1.65-2.09 | 0.43 |

### Example 2

In this example, a patient clinically diagnosed with type 2 diabetes and on medication for control of this condition commenced taking an olive leaf extract. The extract was taken in the form of four capsules as a single dose once a day with a glass of water. The capsules were a commercially available product sold by Comvita Ltd. The polyphenol dose in four OLE capsules was approximately 50 mg of oleuropein and 10 mg of hydroxytyrosol.

The results before and after commencing treatment included:
1. An HbA1c score changing from 52 Mmols to 45 Mmol within 47 days of taking the extract;
2. A loss in weight of 7 kg over the 47-day period;
3. Anecdotal comments by the patient that their general state of wellbeing went from being lethargic and tired to “feeling extremely good at the moment” and having “more energy throughout the day” after 47 days of treatment;
4. A reduction in cholesterol after 47 days treatment;
5. A halt in the taking of prescription medicine, metformin for diabetes management;
6. A reduced circumference in stomach area after 47 days treatment.

The above results clearly illustrate that the olive leaf extract has at least reduced the symptoms of type 2 diabetes such as reducing blood sugar levels (as measured via HbA1c scores), increasing perceived energy levels in the patient and other factors as noted above.

### Example 3

In this example, a further patient with type 2 diabetes also commenced treatment taking an olive leaf extract in the form of four capsules as a single dose once a day with a glass of water. The capsules were a commercially available product sold by Comvita Ltd. The polyphenol dose in four OLE capsules was approximately 50 mg of oleuropein and 10 mg of hydroxytyrosol.

The results before and after commencing treatment included:
1. An HbA1 c score changing from 9.7 (77Mmols) to 7.2 (63Mmols) after 9 months of taking the extract;
2. A loss in weight of 8.5kg over the 9-month time period;
3. Anecdotal comments by the patient that their energy levels are “hugely increased” since commencing taking the olive leaf extract;
4. Elimination of the need to commence insulin injections as was about to happen prior to commencing the trial.

As per Example 2, the above results clearly illustrate that the olive leaf extract has at least reduced the symptoms of type 2 diabetes such as reducing blood sugar levels (as measured via HbA1c scores), increasing perceived energy levels in the patient and other factors as noted above.

Aspects of the methods and uses have been described by way of example only and it should be appreciated that modifications and additions may be made thereto without departing from the scope of the claims herein.

1. A method of reducing the symptoms associated with type 2 diabetes in a subject, by the step of oral administration to the subject of a therapeutically effective amount of a water based extract derived from olive (Oka europaea) leaves, wherein the compounds oleuropein and hydroxytyrosol are present in the extract at a ratio of approximately 0.5 to 2.0 parts hydroxytyrosol to approximately 5 parts oleuropein and wherein the olive leaf extract is produced by immersing the leaves in water at a temperature of approximately 80-100° C. for a time period of 30 to 180 minutes.

2. The method as claimed in claim 1 wherein treatment results in increased glucose tolerance in the subject relative to a measured glucose tolerance in the subject if untreated.

3. The method as claimed in claim 1 wherein treatment results in an at least 1% increase in insulin sensitivity in the subject relative to measured insulin sensitivity in the subject if untreated.

4. The method as claimed in claim 1 wherein treatment results in an at least 1% reduction in HbA1c levels in the subject relative to measured HbA1c levels in the subject if untreated.

5. The method as claimed in claim 1 wherein treatment results in an at least 5% increase in insulin excretion relative to measured insulin excretion in the subject if untreated.

6. The method as claimed in claim 1 wherein treatment results in an at least 5% increase pancreatic β-cell secretion capacity relative measured pancreatic β-cell secretion capacity in the subject if untreated.

7. The method as claimed in claim 1 wherein treatment results in an at least 5% increase in IL-6 cytokine production relative to measured IL-6 cytokine production in the subject if untreated.

8. The method as claimed in claim 1 wherein treatment results in increased IGFBP1 and IGFBP2 cytokine production relative to measured IGFBP1 and IGFBP2 cytokine production in the subject if untreated.

9. The method as claimed in claim 1 wherein the hydroxytyrosol is giucoronidated.

10. (canceled)

11. The method as claimed in claim 9 wherein the leaves prior to immersion are chopped to a size of 15 mm or less.

12. The method as claimed in claim 1 wherein the leaves are processed and stabilised within at least 24 hours of picking.

13. The method as claimed in claim 12 wherein stabilisation occurs before, during, or after extraction by reducing the water activity.

14. The method as claimed in claim 12 wherein mixing the extract with a hydrophilic carrier completes stabilisation.

15. The method as claimed in claim 1 wherein the extract is formulated as: a liquid, a powder, tablets, capsules, as a food, fortified in a food, as a drink, and combinations thereof.

16. The method as claimed in claim 1 wherein the extract is administered to the subject at a rate of approximately 30-150 mg oleuropein per day and approximately 0.3-40 mg hydroxytyrosol per day.

17-32. (canceled)
